(19) United States
(12) Patent Application Publication
(10) Pub. No.: US 2004/0202731 A1
(43) Pub. Date: Oct. 14, 2004

(54) Rosmarinic acid composition

(76) Inventors: Robert T. Gow, Naples, FL (US); Brian Pierce, Thousand Oaks, CA (US); John Pierce, Thousand Oaks, CA (US); William Birdsall, Naples, FL (US)

Correspondence Address: TROUTMAN SANDERS LLP, BANK OF AMERICA PLAZA, SUITE 5200, 600 PEACHTREE STREET , NE, ATLANTA, GA, 30308-2216, US

(21) Appl. No.: 10/818,010

(22) Filed: Apr. 5, 2004

Related U.S. Application Data
(63) Continuation-in-part of application No. 10/407,685, filed on Apr. 8, 2003.

(51) Int. Cl. A61K 31/704; A61K 35/78

(52) U.S. Cl. 424/728; 514/33; 514/26

(57) ABSTRACT

This invention is related to methods and compositions comprising rosmarinic acid. More specifically, the present invention comprises compositions comprising borneol, rosmarinic acid and at least one ginsenoside.

### RELATED APPLICATIONS

This application is a continuation-in-part of U.S. patent application Ser. No. 10/407,685, filed Apr. 8, 2003, which is herein incorporated in its entirety.

### FIELD OF THE INVENTION

This invention is related to a composition for treating heart conditions. More specifically, the present invention is related to a composition for treating heart conditions containing borneol, rosmarinic acid and at least one ginsenoside.

### BACKGROUND OF THE INVENTION

It has long been known that Rosemary (Rosmarinus officinalis) is a beneficial medicinal plant and contains potent antioxidants such as rosmarinic acid, as well as essentional oils, such as borneol.

Ginseng has also been recognized as an important herbal medicine. There are many varieties of ginseng and each variety of the ginseng plant contains many pharmacologically active components. Correctly chosen mixtures of such components often have unexpected beneficial effects.

Many people are looking for ways to achieve healthful benefits from natural products. Natural products provide many of the same physiological effects as do prescription or over-the-counter drugs. Like prescription or over-the-counter drugs, natural products may also provide unwanted side effects due to the presence of compounds in the formulations.

Natural products have long been used by humans and animals. Generally, the natural products have been consumed by orally ingesting a tea, infusion or tincture made from one or more natural products. Sometimes the natural products are chewed or smoked to release the desired active agent compounds from the plant material. Many times, this crude extraction of the active agent compounds, by smoking, chewing, or steeping in water or alcohol, releases all of the compounds that are soluble in the solvent, or combustible products are formed.

Crude extraction methods, such as chewing, infusions or burning, of plant material expose people and animals to compounds in the plant material that cause unwanted side effects and long term problems. For example, exposure to the unwanted compounds or combustion products lead to changes in cells exposed to such compounds or products and even establishment of pathogenic conditions such as cancer. For example, it is a wide spread practice to chew betel quid, a combination of piper betel leaf, the nut of areca catechu and flavoring or other ingredients. Among those who chew betel quid, there are changes to the cells of the oral cavity and an increase in oral cancer. Similar pathologies are seen in people who chew tobacco.

What is needed are compositions of natural products that provide beneficial physiological effects without the presence of naturally occurring compounds that are harmful to the body. What is also needed are combinations of compounds that provide beneficial effects.

### SUMMARY OF THE INVENTION

The present invention comprises compositions and methods for providing combinations of beneficial compounds. Such compositions are provided in pharmaceutical formulation for administration to humans and animals. For example, the compositions may comprise a paste, resin, oil, or a powder suitable for use in a fast dissolve tablet and other applications, containing rosmarinic acid, borneol and at least one ginsenoside such as ginsenoside Rg1, ginsenoside Re, ginsenoside Rd and ginsenoside Rb1. Such products have the unexpected benefit of treating heart and other cardiovascular conditions. The present invention is useful in making red blood cells more elastic, preventing atherosclerosis, improving contractile ability of the heart, lowering blood pressure, inhibiting lipid oxidation, ameliorating the effects of ischemia, improving blood circulation, resolving blood stasis, inhibiting platelet aggregation, and preventing restenosis.

The compositions of the present invention can made by extraction methods that isolate the desired compounds and may be found in the form of a paste, resin, or oil and then processed for various uses. For direct ingestion, for example, a paste can be sweetened and flavored. In addition or alternatively, the paste can be mixed with other dietary supplements, such as the very sweet tasting herb Stevia rebaudiana, flavors, anti-oxidants, and/or extract of llex paraguariensis, as well as other botanical extracts. The compositions may be made by combining the desired isolated compounds to form the combination compositions of the present invention. Some compounds are available from commercial sources.

A paste or other form of the desired compounds of the present invention can also be further processed to prepare a high quality dry flowable powder which can be used, for example, to produce an ingestible tablet. In one embodiment, a paste is combined with a carrier, such as maltodextrin, dextrose, or starches and mixed with a suitable solvent, such as ethyl alcohol or water. The mixture is then spray dried to produce a powder having grains comprising rosmarinic acid, borneol and at least one ginsenoside and the carrier. In a second embodiment, an emulsion of paste is formed in water or ethyl alcohol using, e.g., magnesium carbonate, magnesium carbonate and silica (at up to about 2% by weight), whey protein, maltodextrrin, carboxymethylcellulose and/or other suitable materials. The emulsion is then dried and powdered.

The resulting powder can then be formed into a tablet that, when placed in the mouth, dissolves rapidly over a period of between about 5 seconds to about 120 seconds and preferably in about 15 to about 60 seconds. A tableting powder can be formed by combining between about 18% to about 60% by weight of the powdered composition with between about 30% to about 80% by weight of a dry water-dispersible adsorbant such as magnesium carbonate, or a diluent, such as lactose. Other dry tablet additives, such as one or more of a sweetener, flavoring and/or coloring agents, a binder, such as acacia or gum Arabic, a lubricant, a disintegrant, and a buffer, can also be added to the tableting powder. Preferably, the dry ingredients are screened to a particle size of between about 80 to about 100 mesh.

### DETAILED DESCRIPTION

The present invention comprises compositions and methods of making such compounds for treatment of physiological conditions, such as cardiology conditions. One composition of the present invention comprises rosmarinic acid, borneol and at least one ginsenoside. An aspect of this composition comprises the borneol component comprising about 0.05% to about 85% of the sum of the masses of the three components, more preferably, between about 10% to about 65%. The rosmarinic acid component may comprise about 0.05% to about 85% of the sum of the masses of the three components, more preferably, between about 10% to about 65%. The ginsenoside component may comprise about 0.05% to about 85% of the sum of the masses of the three components, more preferably, between about 10% to about 65%.

According to an aspect of the invention, a paste of the present invention can be further processed to produce a dry, flowable powder. The powder can be used as a dietary supplement and can be added to various edible products. The powder is also suited for use in a rapid dissolve tablet.

According to a particular aspect of the invention, the powder is produced to have a composition that is particularly well suited for delivery in the oral cavity of human subjects, e.g., via a rapid dissolve tablet.

Rosmarinic acid is a naturally-occurring phenolic compound with antioxidant and anti-inflammatory properties. It is currently believed that this compound inhibits lipid peroxidation of rat liver microsomes by 90% at a concentration of 25 μg/ml. Rosmarinic acid suppresses endotoxin-induced activation of complement and concomitant formation of prostacyclin. Formation of 5-HETE and LTB4 from human PMNL is inhibited by rosmarinic acid at concentrations of 10-5 to 10-3 M.

The rosmarinic acid of the present invention can be obtained commercially or can be isolated from plant materials by the methods described herein. Methods for isolating and purifying rosmarinic acid from various sources, such as rosemary and oregano, are known in the art. For example, Oregano (Origanum vulgare) and other medicinal mint species have antioxidant properties which are now known to be due in large part to rosmarinic acid (RA). As an antioxidant, rosmarinic acid prevents cell damage caused by free radical reactions that are thought to be involved in inflammation, degenerative arthritis and the aging process in general RA's antioxidant activity is high.

Rosmarinic acid is categorized as a plant phenolic and is found in a great variety of plants besides oregano and rosemary. Plants such as sanicle, gypsywort, water horehound, lemon balm, the mints, marjoram, and sage contain rosmarinic acid in large amounts, more than 3%, based on dry weight. It is thought that these plants use RA as a defense compound against pathogens and herbivores.

Because it has antimicrobial and antioxidant qualities, and because it can be produced in large quantities by extraction from easily cultivated plants, rosmarinic acid has been commercially applied to food preservation for example, to kill pathogens in sliced meat products. These same properties, plus those of being anti-inflammatory and antiviral, make RA also of interest as a nutritional supplement. It is well absorbed from the gastrointestinal tract and from the skin.

Rosmarinic acid shows promise as a preventative for atherosclerosis because RA and other polyphenols prevent the oxidation of LDL (low density lipoprotein), and oxidized LDL is a primary instigator of plaque formation. Furthermore, this inhibition of LDL oxidation is synergistically enhanced by combining RA with certain other nutritional supplements, such as lycopene.

Rosmarinic acid can also be used in treatments for bronchial asthma, as in Korea, where a high-RA herb called ‘perilla’ is used, diseases related to complement activation, such as rheumatoid arthritis, toxic shock syndromes treatment of peptic ulcer, arthritis, and inflammatory diseases in general and promotion of collagen and elastin synthesis in the skin

Borneol is another component of the compositions of the present invention. Borneol is also known as 1-borneol, endo-1,7,7-trimethyl-bicyclo[2.2.1]heptan-2-ol, molecular formula: C₁₀H₁₈O, CAS No: 507-70-0. Physical data for borneol can be used to isolate the compound and verify its identity. For example the melting point of 207° C., the boiling point of 210° C., and the flash point of 65° C. can be used.

Borneol can be derived by distilling the tree trunk of Dryobalanops aromatica Gaertn. f. (family Dipterocarpaceae) and then cooling the distillate. The extract obtained in this way is called Longnao or Mei borneol (Dryobalanops borneol). Borneol can be synthesized from turpetine oil or camphor. Borneol can also be made from the sublimate of the leaves of Blumea balsamifera DC. (family Compositae). Camphor is also produced by both species, although the D. Aromatica G. is considered superior for both Borneol and Camphor. B. Balsamifera is considered to produce a higher quality Camphor than C. Camphora and is commonly used in Chinese medicine. It contains about 25% Borneol. Borneol is highly unstable and easily turns to Camphor with oxidation. It is said to occur in the camphor tree of Borneo and Sumatra (Dryobalanops camphora) and is also known as Borneo camphor, Malay camphor, and camphol.

One source of the ginsenosides of the present invention can be obtained from American ginseng, Panax quinquefolius, which contains on the order of 30 ginsenosides, a class of saponin derived from plant sterols. Other ginsenoside sources include other species of the genus of Panax, including but not limited to, Panax notoginseng. Ginsenosides as used herein includes, but is not limited to, ginseng saponins, ginsenoside Rg1, ginsenoside Re, ginsenoside Rd, ginsenoside Rc, ginsenoside RB2, and ginsenoside Rb1

A composition of the present invention comprises rosmarinic acid, borneol and at least one ginsenoside. The individual components of such a composition can be obtained by a variety of means, including commercially obtaining purified compounds. The individual components can be obtained from plant sources by extraction of the plant sources. For example, the plant material can be extracted with a water or aqueous solution, an alcohol solution, or a combination of water and alcohol. The plant material may also be extracted used supercritical means alone or in combination with extraction steps using water or alcohol or combination solutions.

A composition of the present invention comprises the following active agents. The formulation is administered to provide an effective amount of the active agents, preferably the administration is using one tablet, of any type, but preferably a rapid dissolve tablet as described herein, once a day.

In general, the compositions of the present invention comprise rosmarinic acid, borneol and at least one ginsenoside. Ginsenosides include, but are not limited to, ginsenoside Rg1, ginsenoside Re, ginsenoside Rb1, and ginsenoside Rd. An aspect of the invention is a composition comprising ginsenosides wherein the amount or concentration of ginsenoside Rb1 is approximately the same as the amount or concentration of ginsenoside Rg1. Compositions are also included in the present invention wherein the amount or concentration of ginsenoside Rb1 is greater than the amount or concentration of ginsenoside Rg1.

An embodiment of the present invention comprises compositions comprising effective amounts of rosmarinic acid, borneol and at least one ginsenoside. An aspect of a composition comprises 50 mg of rosmarinic acid, 1 mg of borneol, and 12-15 mg of ginsenosides. The ginsenosides can be one ginsenosides, or more than one ginsenosides. A composition comprises ginsenosides wherein more than one ginsenoside is found and the ratio of ginsenoside Rb1 to ginsenoside Rg1 is 1:1. A composition comprises ginsenosides wherein more than one ginsenoside is found and the ratio of ginsenoside Rb1 to ginsenoside Rg1 is greater than 1. A composition comprises ginsenosides wherein more than one ginsenoside is found and the ratio of ginsenoside Rb1 to ginsenoside Rg1 is approximately 1:1, and other ginsenosides are also present. A composition comprises ginsenosides wherein more than one ginsenoside is found and the ratio of ginsenoside Rb1 is 8.5, the ratio of ginsenoside Rg1 is 7.5, the ratio of ginsenoside Re is 1.3 and the ratio of ginsenoside Rd is 1.0.

Compositions of the present invention comprise ginsenosides wherein an effective amount of ginsenosides is administered. Ranges of effective amounts for ginsenosides comprise the following ranges, approximately 2 mg to 7 mg of ginsenoside Rg1, approximately 0.015 mg to 0.064 mg ginsenoside Re, approximately 2 mg to 7 mg of ginsenosides Rb1, and approximately 0.2 mg to 0.8 of ginsenoside Rd.

Compositions of the present invention provide effective amounts of ginsenosides in the present compositions comprise from about 1 mg to 20 mg, from about 4 mg to 15 mg, from about 5 mg to about 10 mg, from about 10 to 20 mg, from about 1 to 15 mg and all ranges encompassed therein, and can be administered in any dosage forms for as many administrations as necessary to provide effective pharmacological effects. Effective amounts of borneol comprise amounts in a range from about 0.1 mg to 15 mg, from about 0.1 mg to 2 mg, from about 0.1 mg to 1.5 mg, from about 0.1 mg to 5 mg, from about 2 mg to 14 mg, from about 5 mg to 15 mg. Effective amounts of rosmarinic acid comprise amounts in ranges from about 5 mg to 75 mg, from 10 mg to 60 mg, from 20 mg to 50 mg, from 25 mg to 45 mg, from 30-40 mg and all ranges encompassed therein. Other compounds that may be included in the compositions of the present invention include, but are not limited to, camphor (in ranges from approximately 1 mg to approximately 15 mg); 3,4-dihydroxyphenyllactic acid (in ranges from approximately 10 mg to approximately 60 mg), lithospermic acid A, (in ranges from approximately 10 mg to approximately 60 mg); lithospermic acid B, (in ranges from approximately 10 mg to approximately 60 mg); salvianolic acid A, (in ranges from approximately 10 mg to approximately 60 mg); salvianolic acid B, (in ranges from approximately 10 mg to approximately 60 mg); and tanshinones, (in ranges from approximately 10 mg to approximately 60 mg) such as tanshinones I, II, isotanshinone II_{A}, and cryptotanshinone.

Compositions of the present invention comprise one or more of the active agents in the ranges taught herein. Compositions can be in a pill, tablet or oral-dispersible tablet or pill, but are not limited to those formulations. Compositions of the present invention comprise between 20 mg and 1000 mg active agent, and preferably between 100 mg and 500 mg active agents and include one or more of the following ginsenosides: a) ginsenoside Rg1, with weight percentage between 0.05% and 4.5%, and preferably between 0.25% and 2.75%; and b) ginsenoside Re, with weight percentage between 0.01% and 2.0%, and preferably between 0.04% and 0.775%; and c) ginsenoside Rb1, with weight percentage between 0.05% and 4.5% and preferably between 0.25% and 2.75%; and d) ginsenoside Rd, with weight percentage between 0.01% and 2.0% and preferably between 0.04% and 0.775%. The ratios of the ginsenosides of the present invention can also comprise ratio distributions of the indicated ginsenosides in the following approximate ratio distributions: ginsenoside Rg1(7.5):ginsenoside Re (1.3):ginsenoside Rb1 (8.5):ginsenoside Rd (1.0), or in other words, the weight percentage of Rg1 is approximately 7.5 times that of Rd; the weight percentage of Re is approximately 1.3 times that of Rd; and the weight percentage of Rb1 is approximately 8.5 times that of Rd. The compositions may also have ginsenosides having a combined, total weight percentage between 0.5% and 30.0%, and preferably between 1.5% and 15%. Compositions of the present invention may comprise between 20 mg and 1000 mg active agents, and preferably between 100 mg and 500 mg active agents, which includes borneol in a weight percentage between 0.1% and 3.0%, and preferably between 0.65% and 2.0%. Compositions also comprise between 20 mg and 1000 mg active agents, and preferably between 100 mg and 500 mg active agents, which includes camphor in a weight percentage between 0.1% and 3.0%, and preferably between 0.55% and 2.0%. Compositions comprise between 20 mg and 1000 mg active agents, and preferably between 50 mg and 500 mg active agents, which include tanshinones (tanshinone I, tanshinone II, isotanshinone II_{A} and cryptotanshinone) in a total weight percentage between 0.01% and 65.0%, and preferably between 10.0% and 35.0%.

Compositions of the present invention can comprise 20 mg and 1000 mg active agents, and preferably between 100 mg and 500 mg active agents, which include analogs and polymers of caffeic acid (3,4-dihydroxyphenyllactic acid (also known as Danshensu), rosmarinic acid, lithospermic acid A (and/or as the salt, magnesium lithospermate A), lithospermic acid B (and/or as the salt, magnesium lithospermate B), salvianolic acid A, and salvianolic acid B in a total weight percentage between 0.01% and 65.0%, and preferably between 10% and 35%. Compositions of the present invention also comprise combinations of the active agents taught herein.

Methods of making the compositions of the present invention comprise processes disclosed herein. In general, methods of the present invention comprise methods wherein the plant material, such as rosemary, sage, ginseng, or camphor tree, is extracted, individually or in combinations, using supercritical CO₂ extraction that is preceded by one or more aqueous, alcoholic or hydroalcoholic extractions, supercritical CO₂ extraction that is followed by one or more one or more aqueous, alcoholic or hydroalcoholic extractions extractions, or methods using supercritical CO₂ extraction alone. Additionally, other methods of the present invention comprise extraction of the plant materials using refrigerant chemicals or compressible gasses such as the C-1 through C-4 alkane series or other known extraction methods. Extraction methods may also comprise use of refrigerant chemicals, known in the art and taught in, for example, U.S. Pat. Nos. 6,455,087 and 5,512,285, and each is expressly incorporated in its entirety herein. Refrigerant chemicals include but are not limited to, hydrofluorocarbons (HFCs), hydrochlorofluorocarbons (HCFCs), and/or chlorofluorocarbons (CFCs) such as: HFC-23, HFC-32, HFC-125, HFC-134a, HFC-143a, HFC-152a, R-404a, R-407c, R-410a, HCFC-22, HCFC-123, HCFC-141b, HCFC-142b, R-502, R-11, R-12, and R-113.

Methods for making oral dosage formulations comprise adding the active agent component in a suitable pharmaceutical carrier or dosage form, such as a tablet, capsule, tablet forming powder, capsule filling powder, rapid dissolve tablet, liquid formulations, buccal formulation, and other oral dosage forms. The active agent component comprises at least rosmarinic acid, borneol and at least one ginsenoside. The active agent component can be made by methods of extraction of plant material or can be made by combination of the active agents obtained from commercial sources. As used herein, the extract can be a paste, oil or resin resulting from extraction of one or more plant materials to produce the active agents individually or in combination. The active agents can be provided from commercial or synthetic sources and these active agents are interchangeable and contemplated as the used in the same manner as active agents derived from extraction methods. Thus where the teaching herein is of using the extract, the active agents derived from any source is contemplated.

In one embodiment of a method for producing a powder, the active agents are mixed with a suitable solvent, such as ethyl alcohol or water, along with a suitable food-grade carrier material, such as maltodextrin, dextrose, or starch and the mixture is spray air-dried using conventional techniques to produce a powder having grains of very small particles combined with the food-grade carrier material.

A wide variety of tablet formulations can be made. Preferably, the tablet has a formulation that results in a rapid dissolution or disintegration in the oral cavity. The tablet is preferably of a homogeneous composition that dissolves or disintegrates rapidly in the oral cavity to release the active compounds over a period of about 5 seconds or less to about 120 seconds or more, preferably about 15 to about 60 seconds.

A rapidly dissolving tablet is taught in Pebley, et al. U.S. Pat. No. 5,298,261, which is herein incorporated in its entirety. In general, the patent teaches a tablet that rapidly disintegrates in aqueous solution includes a partially collapsed matrix network that has been vacuum-dried above the collapse temperature of the matrix. The matrix is preferably at least partially dried below the equilibrium freezing point of the matrix. Vacuum drying the tablet above its collapse temperature instead of freeze drying it below its collapse temperature provides a process for producing tablets with enhanced structural integrity, while rapidly disintegrating in normal amounts of saliva. The tablet preferably carries the active agents and the compositions taught herein. The matrix network of the tablet preferably includes a gum, a carbohydrate and the drug. Especially preferred embodiments also include a flavoring, a sweetener and surfactant. The gum is preferably acacia, guar, xanthan, carrageenan or tragacanth gum. The carbohydrate is preferably mannitol, dextrose, sucrose, lactose, maltose, maltodextrin or corn syrup solids.

Various rapid-dissolve tablet formulations known in the art can be used. Representative formulations are disclosed in the U.S. Pat. Nos. 5,464,632, 6,106,861, and 6,221,392, the entire contents of which are expressly incorporated by reference herein. A particularly preferred tableting composition or powder contains about 10% to about 60% by weight of the powder and about 30% to about 60% of a water-soluble diluent. Suitable diluents include lactose, dextrose, sucrose, mannitol, and other similar compositions. Lactose is a preferred diluent but mannitol adds a pleasant, cooling sensation and additional sweetness in the mouth. More than one diluent can be used. A sweetener can also be included, preferably in an amount of between about 3% to about 40% by weight depending on the desired sweetness. Preferred sweetening substances include sugar, saccharin, sodium cyclamate, aspartame, and Stevia extract, used singly or in combination, although other sweeteners could alternatively be used. Flavorings, such as mint, cinnamon, citrus (e.g., lemon or orange), can also be included, preferably in an amount between about 0.001% to about 4% by weight.

Typically, this tableting composition will maintain its form without the use of a binder. However, if needed, various binders are suitable and can be added in an amount of between about 5% to about 15% or as necessary. Preferred binders are acacia or gum Arabic. Alternative binders include sodium alginate, extract of Irish moss, panwar gum, ghatti gum, mucilage of isapol husks, carboxymethylcellulose, hydroxyethylcellulose, methylcellulose, polyvinylpyrrolidone, VEEGUM® (available for T.T. Vanderbilt Co., Inc. of Norwalk, Conn.), larch arabogalactan, gelatin, Kappa carrageenan, copolymers of maleic anhydride with ethylene or vinyl methyl ether.

A tablet according to this aspect of this invention typically does not require a lubricant to improve the flow of the powder for tablet manufacturing. However, if it is so desired, preferred lubricants include talc, magnesium stearate, calcium stearate, stearic acid, hydrogenated vegetable oils, and carbowax in amounts of between about 2% to about 10% by weight.

Similarly, a disintegrant is not expected to be necessary to produce rapid dissolve tablets using the present tablet composition. However, a disintegrant can be included to increase the speed with which a resulting tablet dissolves in the mouth. If desired, between about 0.5% to about 1% by weight of a disintegrant can be added. Preferred disintegrants include starches, clays, celluloses, aligns, gums, crosslinked polymers (including croscarmelose, crospovidone and sodium starch glycolate), VEEGUM® HV, agar, bentonite, natural sponge, cation exchange resins, aliginic acid, guar gum, citrus pulp, sodium lauryl sulphate in an amount of about 0.5% to about 1% of the total mass of the tablet.

It is also generally considered unnecessary to buffer the tablet composition. However, a buffer may be beneficial in specific formulations. Preferred buffering agents include mono- and di-sodium phosphates and borates, basic magnesium carbonate and combinations of magnesium and aluminum hydroxide.

In a preferred implementation, the tableting powder is made by mixing in a dry powdered form the various components as described above, e.g., active ingredients (rosmarinic acid, bomeol and ginsenoside Rg1/Rb1), diluent, sweetening additive, and flavoring, etc. An overage in the range of about 10% to about 15% of the active extract of the active ingredient can be added to compensate for losses during subsequent tablet processing. The mixture is then sifted through a sieve with a mesh size preferably in the range of about 80 mesh to about 100 mesh to ensure a generally uniform composition of particles.

The tablet can be of any desired size, shape, weight, or consistency. The total weight of the active components in the form of a dry flowable powder in a single oral dosage is typically in the range of about 80 mg to about 600 mg. An important consideration is that the tablet is intended to dissolve in the mouth and should therefore not be of a shape that encourages the tablet to be swallowed. The larger the tablet, the less it is likely to be accidentally swallowed, but the longer it will take to dissolve or disintegrate. In a preferred form, the tablet is a disk or wafer of about ⅛ inch to about ¾ inch in diameter and about 0.2 inch to 0.08 inch in thickness, and has a weight of between about 160 mg to about 1,200 mg. In addition to disk, wafer or coin shapes, the tablet can be in the form of a cylinder, sphere, cube, or other shapes.

The foregoing description includes the best presently contemplated mode of carrying out the invention. This description is made for the purpose of illustrating the general principles of the inventions and should not be taken in a limiting sense. This invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

All terms used herein are considered to be interpreted in their normally acceptable usage by those skilled in the art. Patents and patent applications or references cited herein are all incorporated by reference in their entireties.

What is claimed is:

1. A composition, comprising rosmarinic acid, borneol and at least one ginsenoside.

2. The composition of claim 1, wherein the at least one ginsenosides comprises ginsenoside Rg1, ginsenoside Rb1, ginsenoside Re, or ginsenoside Rd.

3. The composition of claim 1, wherein the at least one ginsenoside comprises at least ginsenoside Rb1 and ginsenoside Rg1.

4. The composition of claim 3, wherein the ratio of ginsenoside Rb1 to ginsenoside Rg1 is 1.

5. The composition of claim 3, wherein the ratio of ginsenoside Rb1 to ginsenoside Rg1 is greater than 1.

6. The composition of claim 1, wherein the at least one ginsenoside comprises ginsenoside Rb1, ginsenoside Rg1, ginsenoside Rd, and ginsenoside Re.

7. The composition of claim 6, wherein the ratio of the ginsenosides is ginsenoside Rg1 (7.5) to ginsenoside Rb1 (8.5) to ginsenoside Re (1.3 to ginsenoside Rd (1.0).

8. A composition, comprising rosmarinic acid, borneol and at least one ginsenoside, wherein the at least one ginsenoside is ginsenoside Rb1, ginsenoside Rg1, ginsenoside Re and ginsenoside Rd.

9. The composition of claim 8, wherein the amount of rosmarinic acid comprises from 5 mg to 75 mg.

10. The composition of claim 9, wherein the amount of rosmarinic acid is 50 mg.

11. The composition of claim 8, wherein the amount of borneol comprises from 0.1 to 15 mg.

12. The composition of claim 11, wherein the amount of borneol is 1 mg.

13. The composition of claim 8, wherein the at least one ginsenoside comprises from 1 mg to 20 mg.

14. The composition of claim 13, wherein the at least one ginsenoside comprises from 12 mg to 15 mg.

15. The composition of claim 14, wherein the 12 to 15 mg of ginsenoside comprises from 2 mg to 7 mg of ginsenoside Rg1, from 0.015 mg to 0.064 of ginsenoside Re, from 2 mg to 7 mg of ginsenoside Rb1, and from 0.2 mg to 0.8 mg of ginsenoside Rd.

16. An oral dosage formulation, comprising an active agent component of rosmarinic acid, borneol and at least one ginsenoside.

17. The formulation of claim 16 wherein the rosmarinic acid is between about 0.05% to about 85%, by mass, of the active agent component.

18. The formulation of claim 16, wherein the borneol is between about 0.05% to about 85%, by mass, of the active agent component.

19. The formulation of claim 16, wherein the ginsenoside component is between about 0.05% to about 85%, by mass, of the active agent component.

20. The formulation of claim 16, wherein the at least one ginsenosides comprises ginsenoside Rg1, ginsenoside Rb1, ginsenoside Re, or ginsenoside Rd.

21. The formulation of claim 16, wherein the at least one ginsenoside comprises at least ginsenoside Rb1 and ginsenoside Rg1.

22. The formulation of claim 21, wherein the ratio of ginsenoside Rb1 to ginsenoside Rg1 is 1.

23. The formulation of claim 21, wherein the ratio of ginsenoside Rb1 to ginsenoside Rg1 is greater than 1.

24. The formulation of claim 16, wherein the at least one ginsenoside comprises ginsenoside Rb1, ginsenoside Rg1, ginsenoside Rd, and ginsenoside Re.

25. The formulation of claim 24, wherein the ratio of the ginsenosides is ginsenoside Rg1 (7.5) to ginsenoside Rb1 (8.5) to ginsenoside Re (1.3 to ginsenoside Rd (1.0).

26. A dry flowable powder having the composition of claim 1.

27. An ingestible product comprising the composition of claim 1.

28. The composition of claim 1, wherein the material is a liquid extract containing the active component.

29. The composition of claim 1, wherein the composition is an oil comprising rosmarinic acid, borneol, and at least one ginsenoside.

30. The composition of claim 1, wherein the composition is a powder comprising rosmarinic acid, borneol, and at least one ginsenoside.

31. The composition of claim 16, wherein the composition is an oil comprising the active agent component.

32. The composition of claim 16, wherein the composition is a powder comprising the active agent component.

31. The powder of claim 30, for use as a tableting powder.

32. The powder of claim 30 for use as a capsule filling powder.

33. A tablet formed from the powder of claim 30.

34. A capsule filled with the powder of claim 30.

35. A rapid dissolve tablet comprising, between about 18% to about 60% by weight of the powder of claim 30; between about 30% to about 80% by weight of a water soluble diluent; between about 5% to about 15% by weight of a binder; and between about 3% to about 40% by weight of a sweetener, with the percentages being by weight and totaling 100%.

36. A rapid dissolve tablet, comprising the composition of claim 1.

37. The oral dosage formulation of claim 16, wherein the oral dosage formulation is a rapid dissolve tablet.
